(19) United States
(12) Patent Application Publication
(10) Pub. No.: US 2025/0262306 A1
(43) Pub. Date: Aug. 21, 2025

(54) Crosslinked Organogels for Drug Delivery

(71) Applicants: MASSACHUSETTS INSTITUTE OF TECHNOLOGY, Cambridge, MA (US); TIBET CHEEZHENG TIBETAN MEDICINE CO. LTD, Linzhi (CN)

(72) Inventors: Xuanhe ZHAO, Cambridge, MA (US); Xinyue LIU, Cambridge, MA (US); Xiaoyu CHEN, Cambridge, MA (US); Weibo WANG, Lanzhou (CN)

(21) Appl. No.: 18/857,403

(22) PCT Filed: Apr. 18, 2023
(86) PCT No.: PCT/US2023/065903

§ 371 Date: Oct. 16, 2024

Related U.S. Application Data
(60) Provisional application No. 63/331,955, filed on Apr. 18, 2022.

(51) Int. Cl. A61K 47/34; A61K 9/06

(52) U.S. Cl. CPC A61K 47/34; A61K 9/06

(57) ABSTRACT

This disclosure relates to use of tough organogels in drug delivery. Specifically, this disclosure relates to use of crosslinked elastomer organogels and organogles of polyurethanes modified with one or more lipophilic groups in drug delivery.

### CROSS REFERENCE TO RELATED APPLICATIONS

This application claims priority to U.S. Provisional Patent Application No. 63/331,955, filed Apr. 18, 2022, the contents of which are hereby incorporated by reference in their entirety.

### BACKGROUND OF THE DISCLOSURE

### Field of the Disclosure

This disclosure relates to use of tough organogels in drug delivery. Specifically, this disclosure relates to use of crosslinked elastomer organogels and organogles of polyurethanes modified with one or more lipophilic groups in drug delivery.

### Description of Related Art

Polymer organogels and hydrogels are important materials for applications ranging from drug delivery, tissue engineering, medical implants, wound dressings, and contact lenses to sensors, actuators, electronic devices, optical devices, batteries, water harvesters, and soft robots. Whereas numerous hydrogels and organogels have been developed over the last few decades, there remains a need to develop novel organogel and hydrogel materials and fabrication methods for various applications.

For example, drug delivery is one of the simplest and most common applications of organogels and hydrogels, yet there is an unmet need with respect to the delivery of majority of drugs on the market or in development pipeline. Namely, more than half of the drugs on the market, such as docetaxel, paclitaxel, doxorubicin, trastuzumab (Herceptin®), etc., and in the delivery pipeline are hydrophobic in nature. While hydrogels are widely used for the delivery of hydrophilic drugs, whey are incompatible with the hydrophobic drugs. Because the polymer matrix of the hydrogel is hydrophilic, the hydrophobic drugs usually have very limited loading quantity and homogeneity in hydrogel matrices.

Thus, there is a need for new materials and approaches in drug delivery, particularly for the effective delivery of hydrophobic or lipophilic drugs.

### SUMMARY OF THE DISCLOSURE

One aspect of the disclosure provides an organogel including a polymer component and an organic solvent. In one embodiment, the polymer component includes one or more of crosslinked elastomers (such organogels are referred herein as latex-based organogels). In one embodiment, the polymer component includes a modified polyurethane (PU) including one or more lipophilic groups attached to the polyurethane, optionally through a linker, at a carbamate moiety of the polyurethane (such organogels are referred herein as PU-based organogels).

The organogels of the disclosure are suitable for drug delivery applications. Thus, another aspect of the disclosure includes an organogel of the disclosure as described herein and at least one therapeutic agent or diagnostic agent.

In one aspect, the disclosure provides a method of delivering a therapeutic agent or diagnostic agent to a surface, including contacting the surface with the organogel of the disclosure as described herein. In certain embodiments, the surface is skin of a subject.

The disclosure also provides a method of treating a disease or disorder. Such method includes administering to a subject in need thereof an effective amount of the organogel of the disclosure as described herein, wherein the least one therapeutic agent or diagnostic agent is released from the organogel to the subject.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the compositions and methods of the disclosure, and are incorporated in and constitute a part of this specification. The drawings illustrate one or more embodiment(s) of the disclosure and, together with the description, serve to explain the principles and operation of the disclosure.

FIG. 1A illustrates the organogel according to one embodiment of the disclosure. (L) is a photograph of crosslinked latex elastomer, and (R) is a latex-based organogel obtained by immersing crosslinked latex elastomer of Ex. 1 in cottonseed oil for one hour.

FIG. 1B provides the results of swelling test on the latex-based organogel obtained by immersing crosslinked latex elastomer of Ex. 1 in cottonseed oil for one hour or in cottonseed oil comprising Nile red.

FIG. 2 illustrates diffusion of a model drug (Nile red) from the drug-infused latex-based organogel prepared according to Ex. 1 and Ex. 3 to the pig skin under room temperature and at about 45-72° C.

FIGS. 3A-3B show evaluation of the drug delivery efficiency by gas chromatography-mass spectrometry. (3A) Gas chromatography and (3B) mass spectrometry after applying a asarone-loaded latex-based organogel prepared according to Ex. 1 and Ex. 3 to the pig skin after two hours (m/z=208).

FIGS. 4A-4B provide the results of mechanical tests on the organogel according to one embodiment of the disclosure. (4A) Strain-stress curves of an intact and a notched organogel based on dodecyl PU-PCL of Ex. 2-3. (4B) Photos of the intact organogel based on dodecyl PU-PCL of Ex. 2-3 under tensile test.

### DETAILED DESCRIPTION OF THE DISCLOSURE

Before the disclosed processes and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

In view of the present disclosure, the methods and compositions described herein can be configured by the person of ordinary skill in the art to meet the desired need. The present disclosure provides tough organogels that provide improvements in drug delivery applications.

Thus, one aspect of the disclosure provides organogels including a polymer component and an organic solvent.

In one embodiment, the polymer component includes one or more that includes one or more of crosslinked elastomers as described herein. Such organogels are referred herein as latex-based organogels.

The latex-based organogels of the disclosure as described herein can be prepared from one or more crosslinked elastomers and organic solvent.

In certain embodiments, the one or more of crosslinked elastomers is vulcanized natural rubber. As used herein “natural rubber” refers to a resin comprising polyisoprene, and mostly cis-polyisoprene, and minor impurities of other organic compounds. Natural rubber is produced by a number of various plants in the form of sap that contains the natural rubber in aqueous suspension (also referred as “latex” or “natural rubber latex”). For example, in Hevea brasiliensis (Hevea trees), Ficus elastic (India rubber tree), and Cryptostegia grandiflora (Madagascar rubbervine), the rubber-bearing sap flows freely and is recovered simply by tapping the plant. In other plants, or non-Hevea plants, the rubber-bearing sap is not as accessible because is stored in individual cells contained within the roots or stems that must be broken down by physical or other means.

In certain embodiments, the one or more of crosslinked elastomers is vulcanized synthetic rubber. In certain embodiments, the one or more of crosslinked elastomers is vulcanized polyisoprene resin. For example, in certain embodiments, the polyisoprene resin is cis-polyisoprene resin. In certain embodiments, the one or more of crosslinked elastomers is vulcanized polybutadiene.

In certain embodiments, the one or more of crosslinked elastomers is silicone rubber, ethylene propylene diene monomer (EPDM) rubber, or butyl rubber.

Crosslinked elastomer as used herein includes vulcanized or cured elastomeric material. One of skill in the art recognizes that vulcanization is a process by which the physical properties of the material are improved by subjecting the material to a chemical process including addition of sulfur or other similar curatives, activators, and/or accelerators. Curing agents collectively refer to sulfur vulcanizing agents and vulcanization accelerators. Suitable sulfur vulcanizing agents include, for example, elemental sulfur (free sulfur) or sulfur donating vulcanizing agents, for example amino disulfide, polymeric polysulfide, and sulfur olefin adducts.

The one or more crosslinked elastomers is mixed with the organic solvent and is subjected to conventional plastic molding methods such as injection molding, extrusion molding, deposition molding, filament molding, and hot-calendaring press molding, to form the organogels. The methods of preparation of the latex-based organogels of the disclosure can be widely applied to various commercially available latexes or other resins, using procedures familiar to the person of ordinary skill in the art and as described herein. For example, the latex-based organogels of the disclosure can be prepared according to Examples 1 and 2, and/or analogous synthetic procedures.

In another embodiment, the polymer component includes a modified polyurethane (PU) including one or more lipophilic groups attached to the polyurethane, optionally through a linker, at a carbamate moiety of the polyurethane. Such organogels are referred herein as PU-based organogels.

In general, the polyurethane of the disclosure is a polymer that has good toughness, stability, and biocompatibility in in vivo and in vitro applications. The person of ordinary skill in the art will appreciate that a given polyurethane will often have a variety of molecular weights and structures in a given sample. Unless otherwise indicated, a “molecular weight” as used throughout is “weight-average” molecular weight, Mw. The Mw can be determined using any known technique, such as light scattering, small angle neutron scattering, X-ray scattering, or sedimentation velocity. The structures provided herein represent a weight average structure over the sample of the polymers. The person of ordinary skill in the art will be able to distinguish between different polymers, as having substantially different average molecular weights, or substantially different structures. Thus, in some embodiments, the polyurethane has a Mw of 500 Da to 50 kDa.

In certain embodiments, the polyurethane is a hydrophilic polyurethane. Examples of suitable ether-based hydrophilic polyurethanes include HydroMed™ D1, HydroMed™ D2, HydroMed™ D3, HydroMed™ D4, HydroMed™ D5, HydroMed™ D6, HydroMed™ D7, HydroMed™ D640 and HydroSlip™ C (all available from AdvanSource Biomaterials, Massachusetts, USA). Examples of suitable hydrophilic thermoplastic polyurethane includes HydroThane™ AL25 (available from AdvanSource Biomaterials, Massachusetts, USA).

In certain embodiments, the polyurethane is a hydrophobic polyurethane polyurethane. In certain embodiments, the polyurethane is an amphiphilic polyurethane. In certain other embodiments, the polyurethane is a block copolymer that contains urethane linkage (such as PU-PCL block copolymer).

The polyurethanes of the disclosure requires modification of lipophilic groups onto the backbones of polyurethane. For example, one or more lipophilic groups is introduced at the carbamate (urethane) moiety of the polyurethane. The lipophilic modification can be formed using various lipophilic alkyl and alkenyl chains or steroid derivatives. For example, in certain embodiments, the lipophilic group comprises C₁₀-C₂₄ alkyl, C₁₀-C₂₄ alkenyl, C₁₀-C₂₄ alkoyl, C₁₀-C₂₄ alkenoyl, or a steroid derivative.

In certain embodiments, the one or more lipophilic groups comprises lauryl, palmityl (cetyl), myristyl, stearyl, oleyl, lauroyl, palmitoyl (cetoyl), myristoyl, stearoyl, or oleoyl. In certain other embodiments, the one or more lipophilic groups comprises cholyl, deoxycholyl, or lithocholyl moiety.

The carbamates (urethanes) in the polyurethane can be modified with reactive groups such as isocyanates, isothiocyanates, and sulfonyl chlorides. For example, the polyurethanes can be modified with diisocyanates, diisothiocyanates, or sulfonyl chlorides (such as methylene diphenyl diisocyanate (MDI), toluene diisocyanate (TDI), 1,6-hexane diisocyanate (HDI), 1,4-butane diisothiocyanate, 1,3-propylene diisothiocyanate, p-phenylene diisothiocyanate, etc.) to introduce the linker moieties into the polyurethanes. These remaining reactive groups on the diisocyanates, diisothiocyanates, or sulfonyl chlorides can further react with functional monomers or functional polymers having the hydroxy, amine, thiol, or carboxyl moiety. For example, the remaining reactive groups may react with fatty acids, fatty alcohols, fatty amines, and fatty thiols to provide the modified polyurethanes of the disclosure comprising one or more lipophilic groups. Alternatively, the remaining reactive groups may react with other functional monomers (such as N,N-dimethylacetamide, N-isopropyl acrylamide, methyl methacrylate, etc.) to provide longer linkers or linkers that are configured to attach at least two or more lipophilic groups.

In certain embodiments, the linker is of formula:

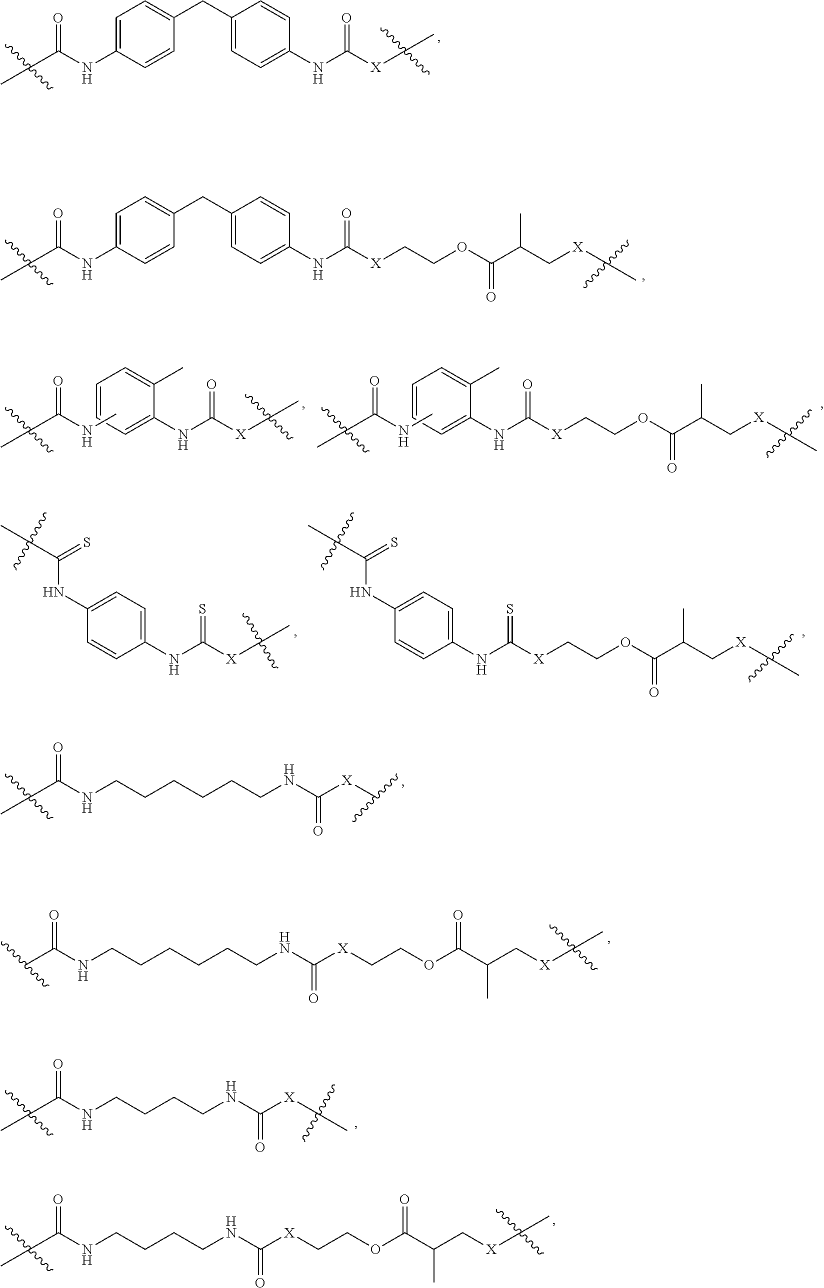

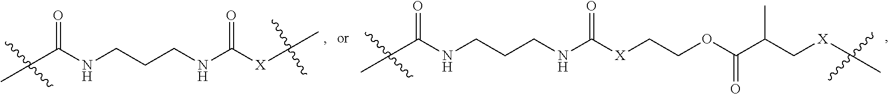

where X is —O—, —S—, or —NH—. In certain embodiments, X is —O—. In certain embodiments, one X is —O—, and the other X is —S—.

The methods of preparation of the modified polyurethanes of the disclosure can be widely applied to various commercially available polyurethanes, under melting or dissolving conditions, using procedures familiar to the person of ordinary skill in the art and as described herein. Many general references providing commonly known chemical synthetic schemes and conditions useful for synthesizing the disclosed modified polyurethanes are available. For example, the modified polyurethanes of the disclosure can be prepared according to general Scheme 1, Examples 2-1 and 2-6, and/or analogous synthetic procedures. One of skill in the art can adapt the reactants and reagents, reaction sequences and general procedures in the examples to fit the desired target molecule. Of course, in certain situations one of skill in the art will use different reactants and reagents to affect one or more of the individual steps or to use protected versions of certain of the substituents. Additionally, one skilled in the art would recognize that compositions of the disclosure can be synthesized using different routes altogether. During any of the processes for preparation of the modified polyurethanes of the disclosure, it may be necessary and/or desirable to protect sensitive or reactive groups on any of the molecules concerned. This may be achieved by means of conventional protecting groups as described in standard works. The protecting groups may be removed at a convenient subsequent stage using methods known from the art.

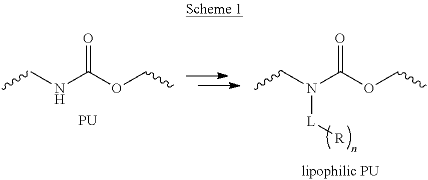

wherein L is a linker, R is a lipophilic group, and n is 1-10000.

The PU-based organogels of the disclosure as described herein can be prepared from the modified polyurethanes and organic solvent. For example, the modified polyurethanes are mixed with the organic solvent and are subjected to conventional plastic molding methods such as injection molding, extrusion molding, deposition molding, filament molding, and hot-calendaring press molding, to form the PU-based organogels.

As provided above, the organogel of the disclosure as described herein includes an organic solvent in addition to the polymer component.

In certain embodiments, the organic solvent of the disclosure as described herein is present in an amount of at least about 10 wt %, or at least about 25 wt %, or at least about 50 wt %, based on total weight of the organogel. In certain embodiments, the organic solvent is present in a range of about 10 wt % to about 80 wt %, based on total weight of the organogel.

Many organic solvents are known in the art. In certain embodiments, organic solvent is a synthetic oil or natural oil, such as cooking oil or vegetable oil. In an example embodiment, the organic solvent is cottonseed oil, avocado oil, canola oil, grapeseed oil, or lavender oil.

The organogels of the disclosure as described herein are considered tough organogels.

The organogels of the disclosure as described herein are considered tough organogels. Such organogels, in certain embodiments, have interfacial toughness of at least 100 J m⁻², or at least 150 J m⁻², or at least 200 J m⁻², or at least 500 J m⁻², or in the range of 700 to 1500 J m⁻², in fully swollen state as measured by, for example, ASTM D 2861 standard 90-degree peeling test.

In certain embodiments, the organogels have a young's modulus values of at least 0.25 MPa, or at least 0.5 MPa, or at least 1 MPa, or at least 2 MPa, or at least 2.5 MPa, or at least 4 MPa, or at least 5 MPa, or at least 10 MPa, as determined by ASTM F2258 tensile test. In certain embodiments, the organogels have rupture stretch value (λ) in the range of 2 to 25, or 2 to 15, or 2 to 10, or 4 to 25, or 4 to 15, or 4 to 10, or 5 to 8, as determined by ASTM F2258 tensile test. In certain embodiments, the organogels have fracture toughness in the range of 2 to 20 KJ/m², as determined by ASTM E1820 tensile test.

The organogels of the disclosure (e.g., the latex-based and the PU-based) can be used in many fields of biomedical engineering. One such application is to use these organogels for lipophilic or hydrophobic drug delivery.

Thus, in certain embodiments, the organogel of the disclosure as described herein further comprises at least one therapeutic agent or diagnostic agent, dispersed within the polymer component. A hydrophobic small molecule drug may be particularly suitable for including in the organogels of the disclosure. Examples of hydrophobic small molecule drug include, but are not limited to, an anti-cancer agent, an antibiotic, an antiviral, an antiparasitic agent, an anti-inflammatory, an anticoagulant, an analgesic agent, an anesthetic agent, an and any combination thereof. A lipophilic dye (such as Nile red) or a fluorescent lipophilic dye (such as Dil) may also be particularly suitable for including in the organogels of the disclosure.

The organogels comprising at least one therapeutic agent or diagnostic agent are suitable for drug delivery applications. Thus, another aspect of the disclosure provides a method of delivering a therapeutic agent or diagnostic agent to a surface, including contacting the surface with the organogel of the disclosure as described herein. In certain embodiments, the surface is skin of a subject.

The organogel comprising at least one therapeutic agent or diagnostic agent as described herein may be used in treating a disease or disorder in a subject. One aspect of the disclosure provides a method of treating a disease or disorder comprising administering to a subject in need thereof an effective amount of the organogel comprising at least one therapeutic agent or diagnostic agent as described herein, wherein the least one therapeutic agent or diagnostic agent is released from the organogel to the subject. In certain embodiments, the administration is topical.

### EXAMPLES

The methods and compositions of the disclosure are illustrated further by the following example, which is not to be construed as limiting the disclosure in scope or spirit to the specific procedures and compounds described in them.

### Example 1. Preparation of the Latex-Based Organogel of the Disclosure

Non-vulcanized natural rubber latex (liquid or solid form) and sulfur w compounded together to form a uniform dispersion. To facilitate the blending, emulsifiers may optionally be added into the mixture. The solidification and vulcanization are completed at room temperature, and residual chemicals are rinsed by a large amount of soapy water.

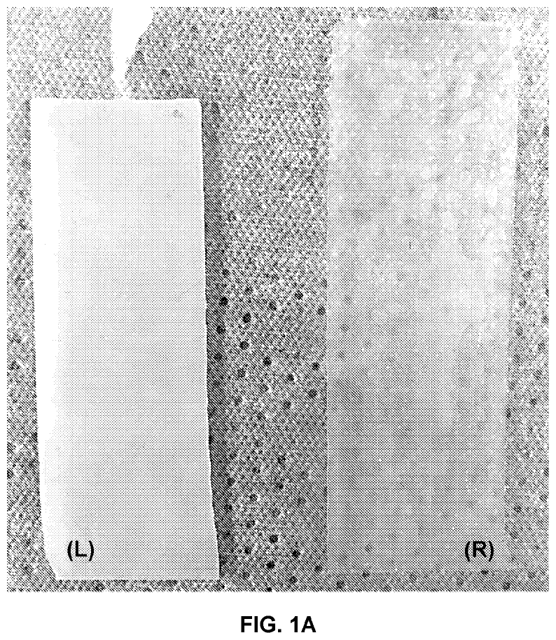

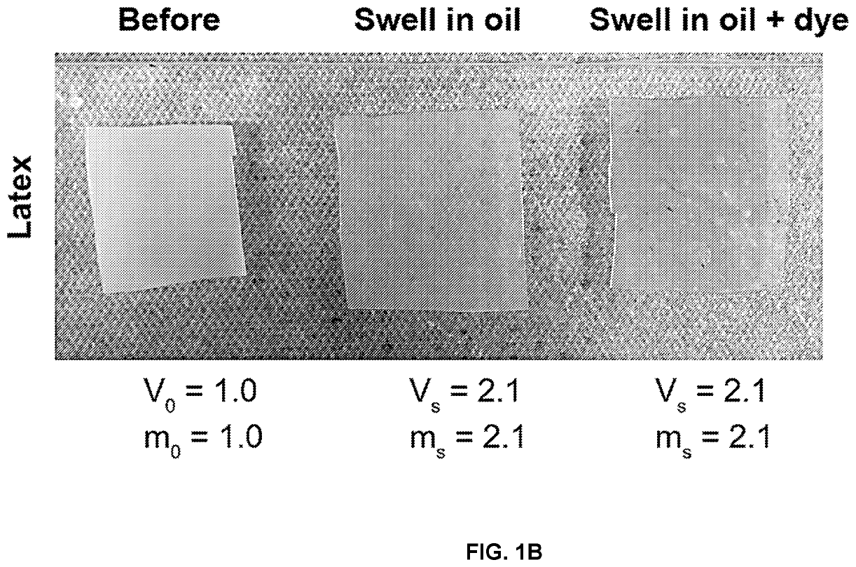

Crosslinked (i.e., vulcanized) natural rubber elastomer is immersed in cottonseed oil for 1-6 hour to obtain the crosslinked elastomer organogel as illustrated in FIG. 1A.

### Example 2. Preparation of the PU-Based Organogel of the Disclosure

### 2-1. Preparation of the Modified Polyurethane of the Disclosure

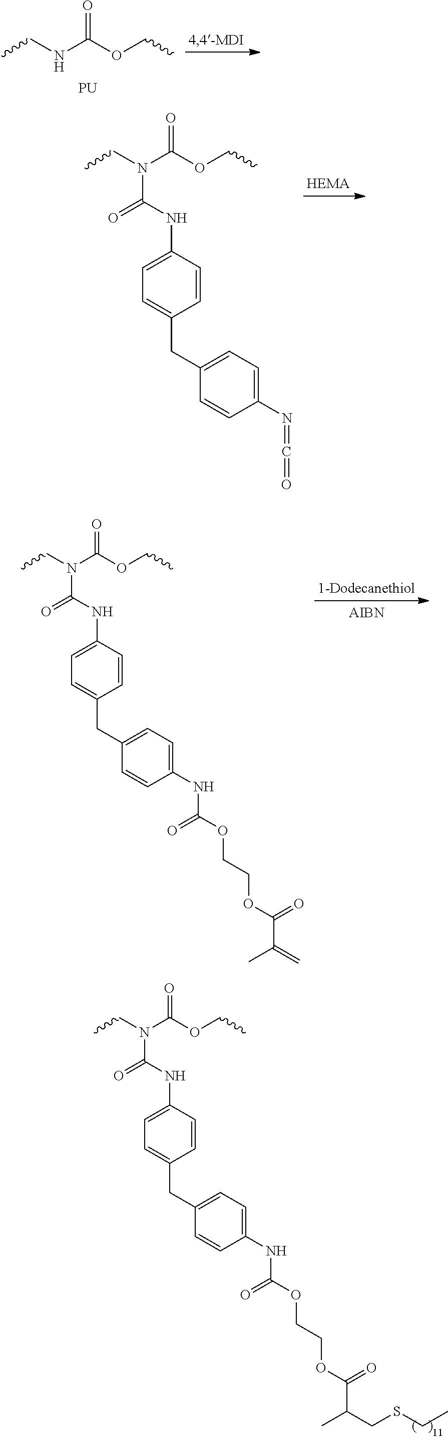

HydroMed™ D640 PU (3 g; available from AdvanSource Biomaterials, Wilmington, MA) was dissolved in anhydrous N, N-dimethylformamide (DMF) (20 mL) in a three-neck flask equipped with a mechanical stirrer under a nitrogen atmosphere. Then, 4,4′-methylenebis(phenyl isocyanate) (4,4′-MDI) (0.2 g; available from Sigma-Aldrich, Inc., St. Louis, MO) was added to the polyurethane solution and stirred for 40 min at 50° C. Next, 2-hydroxyethyl methacrylate (HEMA) (0.3 mL; Sigma-Aldrich, Inc.) was added to the reaction mixture, and the reaction was carried out for one additional hour. 1-Dodecanethiol (2 mL; Sigma-Aldrich, Inc.) and 2,2′-azobis(2-methylpropionitrile) (AIBN) (30 mg; Sigma-Aldrich, Inc.) were subsequently added. The reaction was continued for 3 hours at 70° under mechanical stir. When the reaction was completed, the product was precipitated in ethanol (1.5 L) to terminate the reaction. The product was cut into pieces and thoroughly washed with distilled water and ethanol under magnetic stirring to remove any remaining reactants. The final product was filtered and dried at 65° C. for one day to obtain the lipophilic polyurethane of the disclosure, (4-(4-(((2-((3-(dodecylthio)-2-methylpropanoyl)oxy) ethoxy) carbonyl)amino)benzyl)phenyl) carbamoyl-modified PU (dodecyl PU-D640).

### 2-2. Preparation of the Dodecyl PU-D3

Using the procedure disclosed in Example 2-1, similar modification was carried out using HydroMed™ D3 PU (available from AdvanSource Biomaterials, Wilmington, MA) to obtain (4-(4-(((2-((3-(dodecylthio)-2-methylpropanoyl)oxy)ethoxy)carbonyl)amino)benzyl)phenyl)carbamoyl-modified PU (dodecyl PU-D3).

### 2-3. Preparation of the Dodecyl PU-PCL

Using the procedure disclosed in Example 2-1, similar modification was carried out using MDI-polyester/polyether polyurethane (PU-PCL, poly[4,4′-methylenebis(phenyl isocyanate)-alt-1,4-butanediol/di(propylene glycol)/polycaprolactone], CAS Number: 68084-39-9, purchased from Sigma-Aldrich product Number 430218 to obtain (4-(4-(((2-((3-(dodecylthio)-2-methylpropanoyl)oxy) ethoxy) carbonyl)amino)benzyl)phenyl) carbamoyl-modified PU (dodecyl PU-PCL).

### 2-4. Preparation of the Lipophilic Polyurethane of the Disclosure

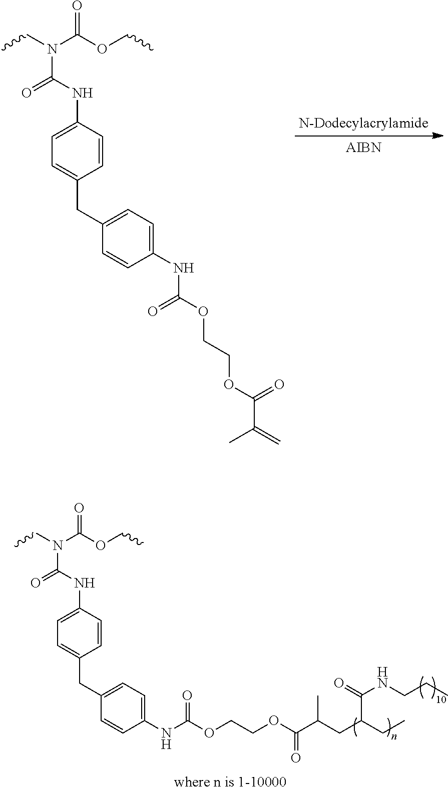

This PU was modified using the procedure in Example 2-1. In short, PU was dissolved in anhydrous DMF in a three-neck flask equipped with a mechanical stirrer under a nitrogen atmosphere. Then, 4,4′-MDI was added to the polyurethane solution and stirred for 40 min at 50° C. Next, HEMA was added to the reaction mixture, and the reaction was carried out for one additional hour. Finally, N-dodecylacrylamide and AIBN were subsequently added, and the reaction was continued for 3 hours at 70° under mechanical stir. When the reaction was completed, the product was worked up as noted.

### 2-5. Preparation of the Modified Polyurethane of the Disclosure

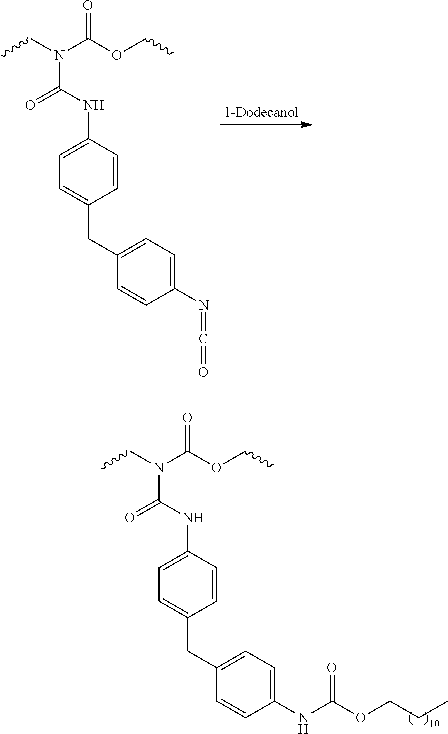

PU was dissolved in anhydrous DMF in a three-neck flask equipped with a mechanical stirrer under a nitrogen atmosphere. Then, 4,4′-MDI was added to the polyurethane solution and stirred for 40 min at 50° C. Next, 1-dodecanol was added to the reaction mixture, and the reaction was carried out to completion.

### 2-6. Preparation of the Modified Polyurethane of the Disclosure

PU was dissolved in anhydrous DMF in a three-neck flask equipped with a mechanical stirrer under a nitrogen atmosphere. Then, 4,4′-MDI was added to the polyurethane solution and stirred for 40 min at 50° C. Next, lauric acid was added to the reaction mixture, and the reaction was carried out to completion.

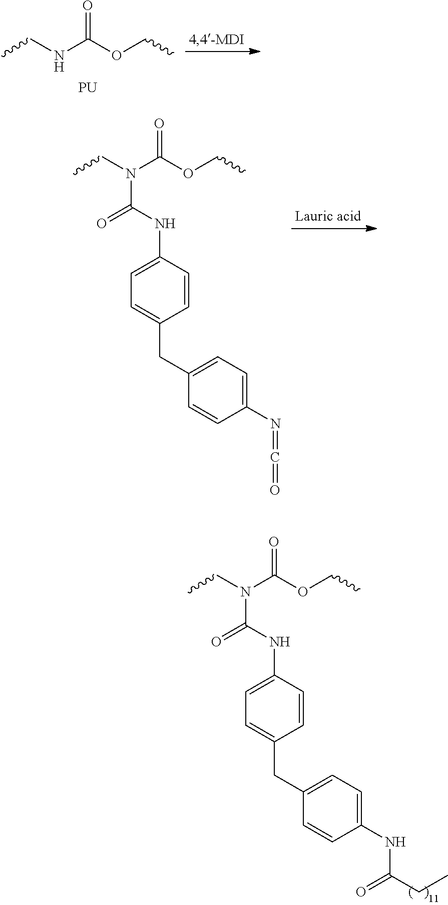

### 2-7. Preparation of the PU-Based Organogels of the Disclosure

The lipophilic polyurethanes of the disclosure are immersed in vegetable oil (or another organic solvent or oils) to form the organogels of the disclosure. For example, the dried polyurethanes obtained in Examples 2-1 to 2-6 were immersed in cottonseed oil for eight hours to prepare their respective organogels.

### Example 3. Preparation of the Drug-Infused Organogels of the Disclosure

A lipophilic drug is mixed in vegetable oil (or another organic solvent) until homogeneous. For example, first, a lipophilic dye Nile red, which is 9-(diethylamino)-5H-benzo[a]phenoxazine-5-one and used as a proof of concept lipophilic drug, was homogeneously dissolved in vegetable oil under mild stir. Then, crosslinked natural rubber elastomer obtained from the procedure of Example 1 or dried lipophilic polyurethanes obtained in Examples 2-1 to 2-6 were immersed in the drug-containing vegetable oil for 1-6 hours to form the organogels of the disclosure.

In certain embodiments and examples, asarone (2,4,5-trimethoxyphenyl-2-propene) was used instead of Nile red. Asarone has significant neuroprotective, antipyretic, analgesic, and anticonvulsant activities for therapeutic applications.

### Example 4. Physical Evaluation of the Organogel of the Disclosure

Swelling test: To measure weight and volume change of the organogels of the disclosure, the “dry” samples (e.g., modified PU) without any solvent were immersed in vegetable oil for eight hours to form organogels. Optionally, Nile red dye (or another lipophilic dye) is included in the vegetable oil as described in Example 3 in order to conveniently visualize of the swelling. The weight and volume change of the polymer component of the disclosure in cottonseed oil are provided in Table 1. The swelling ratio is provided as % change in weight of the elastomer material before and after immersion in oil (e.g., increase in wt % indicates the polymer component absorbs the oil and forms the organogel.)

TABLE 1
Swelling test results of the organogels of the disclosure

| Polymer component | Swelling ratio (wt %) |
|---|---|
| PU-D3* | 100 |
| dodecyl PU-D3 (Ex. 2-2) | 110 |
| PU-PCL** | 100 |
| dodecyl PU-PCL (Ex. 2-3) | 300 |

*unmodified, HydroMed ™ D3 PU;
**unmodified PU-PCL

The results in this table illustrate that, for example, 1 g of dodecyl PU-PCL can absorb 2 g cottonseed oil, while 1 g of dodecyl PU-D3 can absorb 0.1 g cottonseed oil.

The weight change of the vulcanized natural rubber elastomer of Ex. 1 in cottonseed oil are 210 wt % and 210 vol % in swelling ratio as illustrated in FIG. 1B. The results in this table illustrate that, for example, 1 g of crosslinked elastomer can absorb 1.1 g cottonseed oil.

Mechanical test: The mechanical properties of the organogels of the disclosure were tested by standard tensile tests (ASTM F2258) and fracture energy test (ASTM E1820).

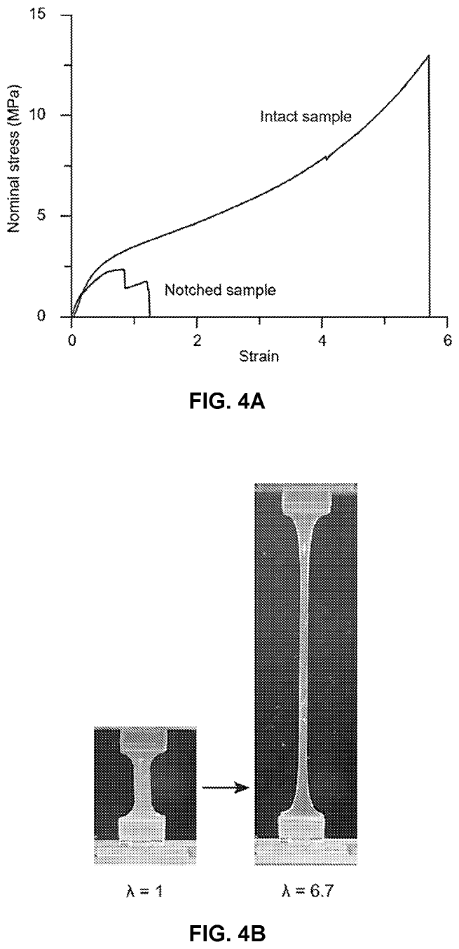

FIGS. 4A-4B provide the results of mechanical testing on organogel obtained by immersing dodecyl PU-PCL (Ex. 2-3) in cotton seed oil for 8 hours. Dodecyl PU-PCL organogel is very elastic (λ=6.7) and shows a high Young's modulus (5.5 MPa) in the tensile test.

### Example 5. Ex Vivo Drug Delivery Test

The drug-infused organogel of the disclosure as described in Ex. 3 was placed on a piece of pig skin. An activated hand warmer (approx. 45-72° C.) was placed on top of the organogel to test for accelerated drug release. The unheated and heated organogel was left in contact with the skin for 1-6 hours. The organogel was then removed and the pig skin was examined for the presence of the drug.

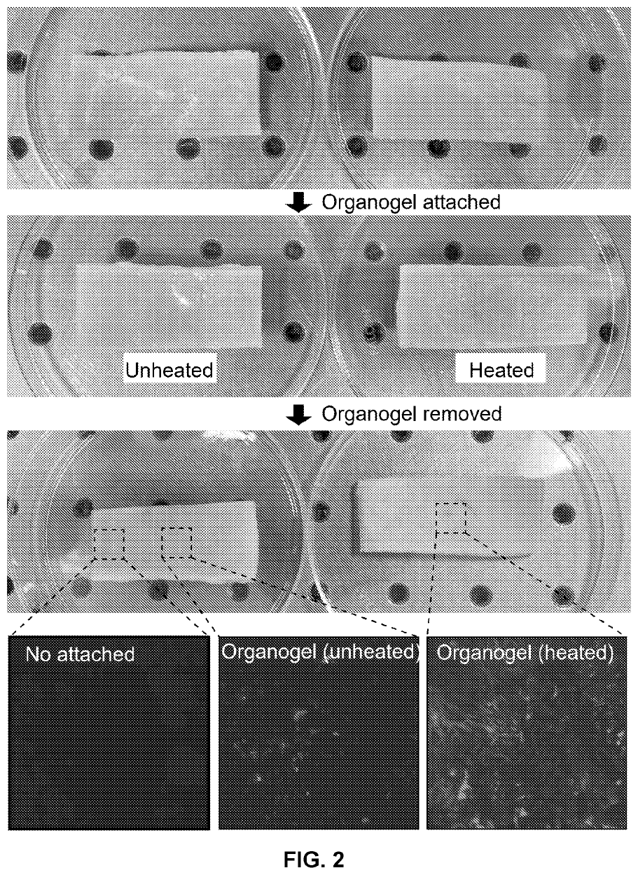

When Nile red dye is used as a model drug, the color change on the pig skin can be visually observed or by microscopy. For example, the results using the Nile red-infused latex-based organogel prepared according to Ex. 1 and Ex. 3 are provided in FIG. 2. As can be seen by visual inspection, the drug-infused organogel has a higher drug delivery when heated than when unheated.

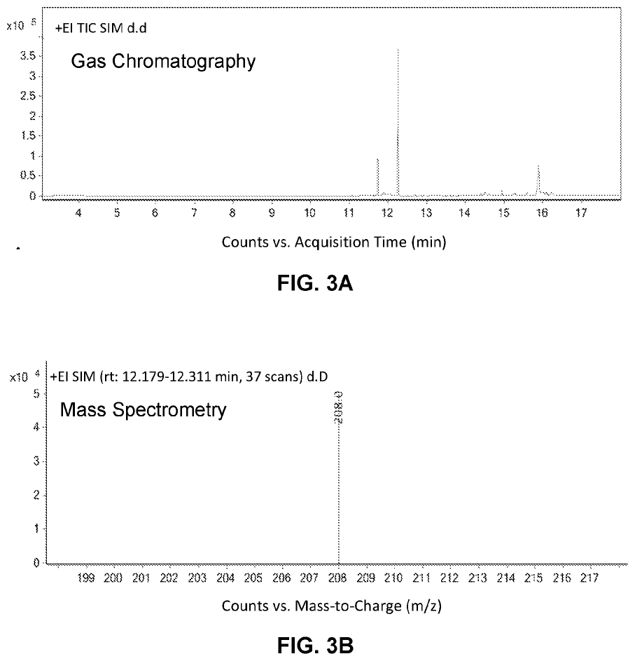

The drug delivery efficiency may also be quantified by the gas chromatography-mass spectrometry (GC-MS) method. For example, after applying the drug-loaded organogel sample on pig skin for 1-6 hours, the organogel was removed and the surface of the skin cleared from oil. The pig skin was then grounded and the skin material extracted with a solvent (such as dichloromethane (DCM), DMF, methanol, etc.). After removing the insoluble particles in the solvent by a filter (0.45 μm), the extracted solvent was injected into the GC-MS to evaluate the drug delivery efficiency of the organogel by using scanning mode and single ion monitoring (SIM) mode. FIGS. 3A-3B illustrate evaluation of the efficiency of delivering asarone by GC-MS from the asarone-loaded latex-based organogel prepared according to Ex. 1 and Ex. 3.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

1. An organogel comprising: a polymer component, an organic solvent, and at least one therapeutic agent or diagnostic agent dispersed within the polymer component, wherein the polymer component comprising-comprises 1) one or more of crosslinked elastomers, or 2) one or more modified polyurethanes, wherein each polyurethane comprises one or more lipophilic groups attached to the polyurethane, optionally through a linker, at a carbamate moiety of the polyurethane.

2. The organogel of claim 1, wherein the organogel comprises at least one therapeutic agent that is a hydrophobic small molecule drug selected from group consisting of an anti-cancer agent, an antibiotic, an antiviral, an antiparasitic agent, an anti-inflammatory, an anticoagulant, an analgesic agent, an anesthetic agent, and any combination thereof, or wherein the organogel comprises at least one diagnostic agent that is a hydrophobic small molecule.

3. (canceled)

4. The organogel of claim 2, wherein the at least one therapeutic agent or diagnostic agent is present in the range of about 5 to 50 wt % based on the weight of the organogel.

5-6. (canceled)

7. The organogel of claim 1, wherein the organogel comprises one or more of crosslinked elastomers as the polymer component and at least one hydrophobic small molecule drug.

8. The organogel of claim 7, wherein the one or more of crosslinked elastomers is vulcanized natural rubber.

9-11. (canceled)

12. The organogel of claim 1, wherein the organogel comprises one or more of modified polyurethanes as the polymer component and at least one hydrophobic small molecule drug.

13. The organogel of claim 12, wherein the modified polyurethane is a hydrophilic polyurethane.

14. The organogel of claim 13, wherein the hydrophilic polyurethane is an ether-based hydrophilic polyurethane, or is a hydrophilic thermoplastic polyurethane elastomer.

15. (canceled)

16. The organogel of claim 12, wherein the polyurethane is an amphiphilic polyurethane.

17. The organogel of claim 12, wherein the polyurethane is a hydrophobic polyurethane.

18. The organogel of claim 12, wherein the polyurethane is a block copolymer that contains urethane linkage.

19. (canceled)

20. The organogel of any of claim 12, wherein the one or more lipophilic groups on the modified polyurethane comprises lauryl, palmityl (cetyl), myristyl, stearyl, oleyl, lauroyl, palmitoyl (cetoyl), myristoyl, stearoyl, or oleoyl, or a cholyl, deoxycholyl, or lithocholyl moiety.

21. (canceled)

22. The organogel of claim 12, wherein the linker on the modified polyurethane is derived from diisocyanates, diisothiocyanates, or sulfonyl chlorides and/or is of the formula: 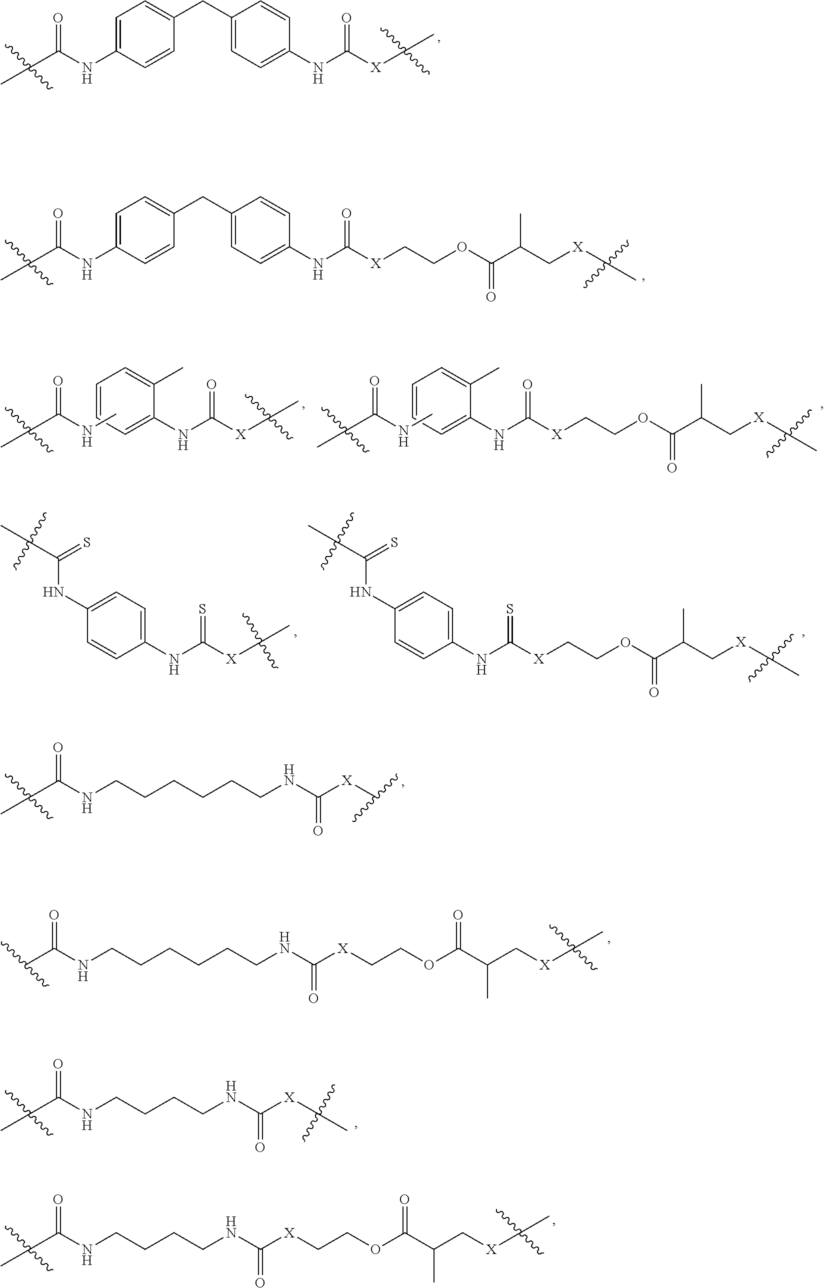 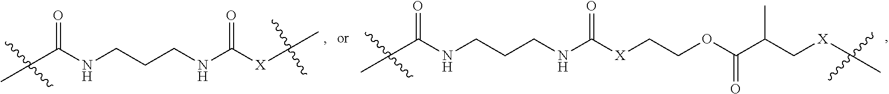
where X is —O—, —S—, or —NH—.

23. (canceled)

24. The organogel of claim 12, wherein the linker on the modified polyurethane is configured to attach one lipophilic group or is configured to attach to 1 to 10000 lipophilic groups.

25. (canceled)

26. The organogel of claim 12, wherein the modified polyurethane has a Mw of 500 Da to 50 kDa.

27. The organogel of claim 1, wherein the organic solvent is present in an amount of at least about 10 wt %, or at least about 25 wt %, or at least about 50 wt %, based on total weight of the organogel, and wherein the organic solvent is a synthetic oil or natural oil.

28-30. (canceled)

31. The organogel of claim 1, having
(a) a young's modulus values of at least 0.25 MPa, of at least 0.5 MPa, of at least 1 MPa, of at least 2 MPa, of at least 2.5 MPa, or at least 4 MPa, or at least 5 MPa, or at least 10 MPa, as determined by ASTM F2258 tensile test;
(b) interfacial toughness of at least 100 J m⁻², or at least 150 J m⁻², or at least 200 J m⁻², or at least 500 J m⁻², or in the range of 700 to 1500 J m⁻², in fully swollen state as determined by ASTM D 2861 standard 90-degree peeling test;
(c) rupture stretch value (2) in the range of 2 to 25, or 2 to 15, or 2 to 10, or 4 to 25, or 4 to 15, or 4 to 10, or 5 to 8, as determined by ASTM F2258 tensile test; and/or
(d) fracture toughness in the range of 2 to 20 KJ/m², as determined by ASTM E1820 tensile test.

32-36. (canceled)

37. A method of delivering a therapeutic agent or diagnostic agent to a surface, comprising contacting the surface with the organogel of claim 1.

38. The method of claim 37, wherein the surface is the skin of a subject.

39. (canceled)

40. A method of treating a disease or disorder, comprising administering to a subject in need thereof an effective amount of the organogel of any of claim 1, wherein the least one therapeutic agent or diagnostic agent is released from the organogel to the subject.
